(19) United States
(12) Patent Application Publication
(10) Pub. No.: US 2023/0028043 A1
(43) Pub. Date: Jan. 26, 2023

(54) MICROBIAL COMBINATIONS WITH MODULATORS OF THE OPIOID SYSTEM AND USES THEREOF

(71) Applicant: EYAL RESEARCH CONSULTANTS LTD., JERUSALEM (IL)

(72) Inventors: AHARON EYAL, JERUSALEM (IL); NOA RAZ, GIZO (IL)

(21) Appl. No.: 17/885,620

(22) Filed: Aug. 11, 2022

Related U.S. Application Data
(63) Continuation-in-part of application No. PCT/IB2021/051113, filed on Feb. 11, 2021.
(60) Provisional application No. 62/975,807, filed on Feb. 13, 2020.

(51) Int. Cl. A61K 35/747; A61K 31/4468; A61K 31/485; A61K 45/06; A61K 35/745; A61K 35/741

(52) U.S. Cl. CPC A61K 35/747; A61K 31/4468; A61K 31/485; A61K 45/06; A61K 35/745; A61K 35/741

(57) ABSTRACT

Treating, and compositions for treating, a condition and/or a symptom associated with an effect of at least one element of the opioid system in an animal by administering to the animal (i) a therapeutically effective amount of at least one microorganism and/or of a prebiotic thereof, where the microorganism is capable of modulating an effect of at least one element of the opioid system of the animal and (ii) a therapeutically effective amount of at least one compound capable of modulating an effect of an element of the opioid system of the animal.

### FIELD

The field of art to which this invention generally pertains is compositions comprising a microorganism and/or a prebiotic thereof capable of modulating an effect of at least one element of the opioid system of an animal and a compound capable of modulating an effect of at least one element of the opioid system of the animal.

### BACKGROUND

The opioid system controls pain, reward and addictive behaviors. Opioids exert their pharmacological actions through opioid receptors, which are found principally in the central and peripheral nervous system and the gastrointestinal tract. Opioid receptors are activated by a family of endogenous peptides which are released by neurons. These receptors mediate both the psychoactive and the somatic effects of opioids. Opioid receptors can also be activated exogenously by alkaloid opiates, the prototype of which is morphine, which remains the most valuable painkiller in contemporary medicine.

Side effects of opioids may include itchiness, sedation, nausea, respiratory depression, constipation, and euphoria. Long-term use can cause tolerance, meaning that increased doses are required to achieve the same effect, as well as physical dependence, meaning that abruptly discontinuing the drug leads to unpleasant withdrawal symptoms. The euphoria attracts recreational use and frequent, escalating recreational use of opioids typically results in addiction. An overdose or concurrent use with other depressant drugs like benzodiazepines commonly results in death from respiratory depression. The reduction of opioids use is a primary goal worldwide.

Probiotics are live microorganisms used for restoring the flora in the body of an animal.

### SUMMARY

According to an aspect of some embodiments of the invention, there is provided a method of treating a condition and/or a symptom associated with an effect of at least one element of the opioid system in an animal, comprising administering to said animal (i) a therapeutically effective amount of at least one microorganism and/or of a prebiotic thereof, wherein said microorganism is capable of modulating an effect of at least one element of the opioid system of said animal and (ii) a therapeutically effective amount of at least one compound capable of modulating an effect of at least one element of the opioid system of said animal.

According to a further aspect of some embodiments of the invention, there is provided a composition comprising

(i) a therapeutically effective amount of at least one microorganism and/or of a prebiotic thereof, wherein said microorganism is capable of modulating an effect of at least one element of the opioid system of an animal, and
(ii) a therapeutically effective amount of at least one compound capable of modulating an effect of at least one element of the opioid system of said animal.

### DETAILED DESCRIPTION

The invention relates to combinations of a microorganism and/or a prebiotic thereof which is capable of modulating an effect of at least one element of the opioid system of an animal with a compound capable of modulating an effect of at least one element of the opioid system of said animal, and to uses thereof in therapy.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “prebiotic” refers to a substrate that is selectively utilized by particular microorganisms conferring a health benefit.

As used herein, the term “probiotic” refers to a live microorganism that, when administered to an animal in adequate amounts, confers a health benefit on the animal.

As used herein, the term “modulating an effect” refers to altering, such as increasing or decreasing a physiological effect of an element.

According to some embodiments, the probiotic and the compound modulate the same element. According to some embodiments, the probiotic and the compound have the same modulatory effect on the same element e.g. both increase the activity and/or expression of the same element or both decrease the activity and/or expression of the same element. According to some embodiments, the probiotic and the compound have different modulatory effects on the same element e.g. the probiotic increases the activity and/or expression of the same element and the compound decreases the activity and/or expression of the same element, or vice versa.

According to some embodiment, the probiotic and the compound modulate different elements.

According to some embodiments, the probiotic and the compound modulate the same element, wherein said same element refers to the same particular element. According to some embodiments, the probiotic and the compound modulate the same element, wherein said same element refers to the same category of elements (e.g. the group of opioid receptors, the group of opioids). According to some embodiments, modulating an effect is achieved by modulating (by increasing or decreasing) expression of an element. According to some embodiments, modulation is achieved by increasing or decreasing a physiological effect of an element.

As used herein, the term “direct modulation” refers to directly acting on an element of the opioid system in order to modulate an effect of that element.

As used herein, the term “indirect modulation” refers to indirectly acting on an element of the opioid system in order to modulate an effect of that element, e.g. by reducing or increasing expression of the element.

As used herein, the term “animal” refers to any animal, including a human being.

As used herein, the term “neuromodulator” refers to is a compound released from a neuron that affects groups of neurons, or effector cells that have the appropriate receptors. The term “neuromodulator” as used herein also refers to a compound released from a cell other than a neuron, which affects the activation of a neuron.

As used herein, the term “element of the opioid system” refers to at least one selected from the group consisting of an endogenous opioid, an opioid receptor and an enzyme responsible for the synthesis or degradation of an opioid.

As used herein, the term “treating” includes ameliorating, mitigating, and reducing the instances of a disease or condition, or the symptoms of a disease or condition.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

As used herein, the term “prodrug” refers to a biologically inactive compound which can be metabolized in the body to produce a drug.

As used herein, the term “opioid receptor” refers to any type of opioid receptors.

As used herein, the term “Opioid” refers to any substance, both natural and synthetic, that binds to opioid receptors (including full agonists, partial agonists, full antagonists and partial antagonists).

As used herein, the term “Opiate” refers to an alkaloid compound naturally found in the opium poppy plant.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Aspects and embodiments of the invention are described in the specification hereinbelow and in the appended claims.

The particulars shown herein are by way of example and for purposes of illustrative discussion of the various embodiments of the invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the invention, there is provided a method of treating a condition and/or a symptom in an animal, comprising administering to said animal (i) a therapeutically effective amount of at least one microorganism and/or of a prebiotic thereof, wherein said microorganism is capable of modulating an effect of at least one element of the opioid system of said animal and (ii) a therapeutically effective amount of at least one compound capable of modulating an effect of at least one element of the opioid system of said animal.

According to an aspect of some embodiments of the invention, there is provided (i) a therapeutically effective amount of at least one microorganism and/or of a prebiotic thereof, wherein said microorganism is capable of modulating an effect of at least one element of the opioid system of an animal and (ii) a therapeutically effective amount of at least one compound capable of modulating an effect of at least one element of the opioid system of said animal.

According to an embodiment, said microorganism and said at least one compound are capable of modulating a same said at least one element of said opioid system of said animal.

According to an embodiment, said microorganism is capable of modulating a first said element of said opioid system and said at least one compound is capable of modulating a second element of said opioid system, wherein said first element is different from said second element.

According to an embodiment, the at least one compound is capable of modulating said element, in some embodiments by direct modulation and in some embodiments by indirect modulation. According to one embodiment, said at least one microorganism is already present in the animal prior to administration. According to one such embodiment wherein said at least microorganism is already present in the animal, administration of said organism provides a further amount of said microorganism to said animal.

According to one such embodiment wherein said at least microorganism is already present in the animal, a prebiotic thereof is administered, thereby facilitating growth of the microorganism. According to one embodiment, said at least one microorganism is not present in the animal prior to administration.

According to an embodiment, said condition and/or symptom is associated with the gastrointestinal tract.

According to an embodiment, said condition and/or symptom is associated with autism spectrum disorder. According to an embodiment, said condition and/or symptom associated autism spectrum disorder is selected from the group consisting of restlessness, rage attack, agitation, treatment-resistant self-injurious behavior, stress, aggression, anxiety, irritability, behavioral outbreaks, communication problems, disruptive behavior, psychosis, hyperactivity, lethargy, seizure activity, hyper sensitivity, stereotypy, inappropriate speech, sleep problem, cognitive impairment, motor and/or vocal tics, digestive problems, lack of appetite, constipation, depression, incontinence, difficulty in concentration, attention disorder and combinations thereof.

According to an embodiment, said condition and/or symptom is associated with central opioid deficiency, endorphin deficiency or both.

According to an embodiment, said condition and/or symptom is associated with pain, depression, seizures, stress, appetite, addiction, abuse and combinations thereof.

According to an embodiment, said element is present in the gastrointestinal tract.

According to an embodiment, said element of the opioid system is selected from the group consisting of opioid receptors, endogenous opioids, opioids synthesizing enzymes, opioids degrading enzymes, and combinations thereof.

According to an embodiment, said element of the opioid system is selected from the group consisting of mu opioid receptors, delta opioid receptors, kappa opioid receptors, nociceptin opioid peptide receptors, sigma opioid receptors, zeta opioid receptors, opioids, dynorphins, enkephalins, endorphins, endomorphins, nocieptin, Dipeptidyl peptidase IV, aminopeptidases, Cytochrome P450 2D6 (CYO2D6)), morphine-6-glucuronide (M6G), morphine-3-glucuronide (M3G), corticotroponin-releasing hormones, proopiomelanocortin, endorphins synthesizing enzymes, endorphins degradation enzymes, endomorphins synthesizing enzymes, endomorphins degradation enzymes, dynorphins synthesizing enzymes, dynorphins degradation enzymes, enkephalins synthesizing enzymes, enkephalins degradation enzymes, nocieptin synthesizing enzymes, nocieptin degradation enzymes and combinations thereof.

According to an embodiment, said modulating an effect of at least one element comprises modulating the expression of at least one element selected from the group consisting of opioid receptors, endogenous opioids, opioids synthesizing enzymes, opioids degrading enzymes and combinations thereof.

According to an embodiment, said modulating an effect of at least one element comprises modulating the activity of at least one element selected from the group consisting of opioid receptors, endogenous opioids, opioids synthesizing enzymes, opioids degrading enzymes and combinations thereof.

According to an embodiment, said modulating an effect of at least one element comprises activating opioid receptors, inactivating opioid receptors, modulating the expression of endogenous opioids, modulating the expression of opioids synthesizing enzymes, modulating the expression of opioids degrading enzymes, modulating the action of another opioid at the opioid receptors and combinations thereof.

According to an embodiment, said microorganism changes the expression of mu opioid receptors.

According to an embodiment, said microorganism changes the expression of endogenous opioids.

According to an embodiment, said compound is an agonist of an opioid receptor

According to an embodiment, said compound is an agonist of a GI tract opioid receptor.

According to an embodiment, said compound is an antagonist of an opioid receptor.

According to an embodiment, said compound is an antagonist of a GI tract opioid receptor.

According to an embodiment, said compound is an allocentric modulator of an opioid receptor.

According to an embodiment, said compound is an allocentric modulator of a GI tract opioid receptor.

According to an embodiment, said compound is a terpene.

According to an embodiment, said compound is a terpene selected from the group consisting of pinene, linalool, caryophyllene, eucalyptol, terpineol cymene, terpinene, phellandrene, eugenol and combination thereof.

According to an embodiment, said compound is an opioid, and/or an opiate.

According to an embodiment, said compound is a morphine, a morphine derivative and/or a morphine prodrug.

According to an embodiment said compound is selected from the group consisting of heroin, codeine, thabine, Fentanyl, hydrocodone, hydromorphone, Dihydrocodeine, oxymorphol, oxycodone, oxymorphone, metopon, nicocodeine, nicomorphine, dihydrocodeine, dihydromorphine, naloxone, naltrexone, diprenorphine, Meperidine, Methadone and combinations thereof.

According to an embodiment, said compound changes the expression of endogenous opioids.

According to an embodiment, said treating induces the production of neurotransmitters and/or neuromodulators in the enteric nervous system.

According to an embodiment, said condition and/or symptom is selected from the group consisting of visceral sensation, visceral pain, visceral and/or gastrointestinal motility and combinations thereof.

According to an embodiment, said microorganism comprises a probiotic microorganism.

According to an embodiment, said at least one microorganism is an anaerobic microorganism.

According to an embodiment, said at least one microorganism is an aerobic microorganism.

According to an embodiment, said microorganism comprises a strain selected from the group consisting of Lactobacillus, Bifidobacterium, Prevotella, Bacteroides and combinations thereof.

According to an embodiment, said microorganism is selected from the group consisting of Lactobacillus rhamnosus, Lactobacillus crispatus, Lactobacillus iners, Lactobacillus gasseri, Lactobacillus jensenii, Lactobacillus acidophilus, Lactobacillus plantarum, Lactobacillus vaginalis, Lactobacillus casei, Lactobacillus reuteri, Lactobacillus salivarius, Lactobacillus Bifidobacterium lactis, Streptococcus thermophilus, Bifidobacterium longum, Bifidobacterium breve, Bifidobacterium infantis, Lactobacillus helveticus, Lactobacillus rhamnosus, Clostridium, Akkermansia, Bacteroides, Parabacteriodes, Actinobacteria, Proteobacteria E. coli, Enterococcus, Prevotella, Corprococcus, and Veilonellaceae, Bacteroides. fragilis and combinations thereof.

According to an embodiment, said method further comprises feeding said animal a high-fat diet.

According to an embodiment, said method further comprises feeding said animal a low-fat diet.

According to an embodiment, said method further comprises administering a component selected from the group of heat-shock protein 90 (Hsp9)0 modulators, extracellular-signal-regulated kinase 1 (ERK1) modulators, extracellular-signal-regulated kinase 2 (ERK2) modulators, Jun N-terminal kinase mitogen-activated protein kinase (JNK MAPK) modulators, p38 MAPK modulators, toll-like receptor 4 (TLR4) modulators, norepinephrine reuptake modulators, serotonin reuptake modulators, cholecystokinin (CCK) modulators, neurokinin modulators and combination thereof.

According to an embodiment, administering said microorganism comprises feeding a food comprising a therapeutically effective amount of said microorganism.

According to an embodiment, said administering of said microorganism is conducted prior to or simultaneously with said administering said compound.

According to an embodiment wherein said microorganism is administered simultaneously with said compound, said microorganism and said compound are provided in the same dosage form. According to an embodiment wherein said microorganism is administered simultaneously with said compound, said microorganism and said compound are provided in separate dosage form.

According to an embodiment, the method comprises multiple administrations of said microorganism and/or said prebiotic and multiple administrations of said compound. According to an embodiment, the method comprises administration of said microorganism and/or said prebiotic four times per day, three times per day, twice per day, once per day, three times per week, twice per week, once per week, twice per month, once per month. According to an embodiment, the method comprises administration of said compound four times per day, three times per day, twice per day, once per day, three times per week, twice per week, once per week, twice per month, once per month.

According to an embodiment, the amount of said at least one compound administered with said microorganism is less than the amount of said at least one compound which would be administered in the absence of said microorganism in order to achieve a same therapeutic effect. According to an embodiment, the amount of said at least one compound administered with said microorganism is about 90%, about 80%, about 75%, about 70%, about 60%, about 50%, about 40%, about 30%, about 25%, about 20%, about 10% or even about 5% of that which would be administered in the absence of said microorganism in order to achieve a same therapeutic effect.

According to a further aspect of some embodiments of the invention, there is provided a composition comprising

(i) a therapeutically effective amount of at least one microorganism and/or of a prebiotic thereof, wherein said microorganism is capable of modulating an effect of at least one element the opioid system of an animal, and
(ii) a therapeutically effective amount of at least one compound capable of modulating an effect of at least one element of the opioid system of said animal.

According to a further aspect of some embodiments of the invention, there is provided at least one microorganism and/or a prebiotic thereof, wherein said microorganism is capable of modulating an effect of at least one element of an opioid system of an animal, for use in therapy in combination with at least one compound capable of modulating an effect of at least one element of the opioid system of said animal.

According to a further aspect of some embodiments of the invention, there is provided a kit comprising at least one microorganism and/or a prebiotic thereof, wherein said microorganism is capable of modulating an effect of at least one element of an opioid system of an animal, and at least one compound capable of modulating an effect of at least one element of the opioid system of said animal.

According to an embodiment, said modulating the effect of said at least one element is a direct modulation.

According to an embodiment, said modulating the effect of said at least one element is an indirect modulation.

According to an embodiment, said at least one microorganism is provided in a dried form. According to one such embodiment, the microorganism is provided in a freeze-dried form.

According to an embodiment, said element is selected from the group consisting of opioid receptors, endogenous opioids, opioids, opioids synthesizing enzymes, opioids degrading enzymes and combinations thereof.

According to an embodiment, said composition induces the production of neurotransmitters and/or neuromodulators in the enteric nervous system.

According to an embodiment, the composition as disclosed herein is for use in treating a condition selected from the group consisting of visceral sensation, visceral pain, visceral and/or gastrointestinal motility and combinations thereof.

According to an embodiment, said microorganism comprises a probiotic.

According to an embodiment, said microorganism comprises a strain selected from the group consisting of Lactobacillus, Bifidobacterium, Prevotella, Bacteroides and combinations thereof.

According to an embodiment, said microorganism is selected from the group consisting of Lactobacillus rhamnosus, Lactobacillus crispatus, Lactobacillus iners, Lactobacillus gasseri, Lactobacillus jensenii, Lactobacillus acidophilus, Lactobacillus plantarum, Lactobacillus vaginalis, Lactobacillus casei, Lactobacillus reuteri, Lactobacillus salivarius, Lactobacillus Bifidobacterium lactis, Streptococcus thermophilus, Bifidobacterium longum, Bifidobacterium breve, Bifidobacterium infantis, Lactobacillus helveticus, Lactobacillus rhamnosus, Clostridium, Akkermansia, Bacteroides, Parabacteriodes, Actinobacteria, Proteobacteria E. coli, Enterococcus, Prevotella, Corprococcus, and Veilonellaceae, Bacteroides. fragilis and combinations thereof.

According to an embodiment, said compound is an agonist of an opioid receptor.

According to an embodiment, said compound is an agonist of a GI tract opioid receptor.

According to an embodiment, said compound is an antagonist of an opioid receptor.

According to an embodiment, said compound is an antagonist of a GI tract opioid receptor.

According to an embodiment, said compound is an allocentric modulator of a GI tract opioid receptor.

According to an embodiment, said compound is a terpene.

According to an embodiment, said compound is a terpene is selected from the group consisting of pinene, linalool, caryophyllene, eucalyptol, terpineol, cymene, terpinene, phellandrene, eugenoland combinations thereof.

According to an embodiment, said compound is an opioid, and/or an opiate.

According to an embodiment, said compound is selected from the group consisting of morphine, a morphine derivative, a morphine prodrug and combinations thereof.

According to an embodiment, said compound is selected from the group consisting of heroin, codeine, thabine, Fentanyl, hydrocodone, hydromorphone, Dihydrocodeine, oxymorphol, oxycodone, oxymorphone, metopon, nicocodeine, nicomorphine, dihydrocodeine, dihydromorphine, naloxone, naltrexone, diprenorphine, Meperidine, Methadone and combinations thereof.

According to an embodiment, the composition further comprises a carrier. According to an embodiment, the composition comprises at least 2% by weight carrier, at least 3%, at least 5%, at least 10%, at least 15%, at least 20%, at least 25%, at least 30%, at least 35% or at least 40%, at least 50%, at least 60%, at least 70%, or at least 80% by weight carrier. Any compound other than opioids, opioid system modulators and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. As used herein, the term “cellulose” refers to cellulose, hemicellulose and their combinations.

According to an embodiment, the composition comprises at least one terpene. The term “terpene”, as used herein, refers to both terpenes and terpenoids.

According to an embodiment, the at least one terpene is selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol isomers thereof and combinations thereof.

According to an embodiment, the composition further comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the composition comprises a surfactant selected from the group consisting of phospholipids, glycerides, glycolipids and combinations thereof. According to an embodiment, the composition further comprises a food-approved texturizer. According to an embodiment, the composition further comprises at least 10 ppm ethanol. According to an embodiment, the composition further comprises at least one of vitamin C, vitamin E, polyunsaturated fatty acids, beeswax and coconut oil. According to an embodiment, the composition further comprises a sweetener.

According to an embodiment, said conditions and/or symptoms associated with autism and specific compositions associated therewith are as disclosed in PCT/IB2020/050635, which is incorporated by reference as if fully set out herein.

According to an embodiment, said composition is provided in a form selected from the group consisting of medical patches, stickers, topicals, creams, varnishes, sprays, edibles, beverages, suppositories, nasal preparations, inhalation mask, sub-lingual tabs, lozenges, chewing gums, preparations containing micro and/or nano-emulsions, preparations containing micro and/or nano-particles and combinations thereof.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only.

### Exemplary Compositions

| Form | Microorganism | Compound | Therapeutic effect |
|---|---|---|---|
| Tablet | Lactobacillus | Linalool + | Depression |
| | rhamnosus | Caryophyllene | |
| Tablet | Bacteroide fragilis + | Morphine | Visceral pain |
| | Clostridium | | |
| Gel | Bifidobacterium | Diprenorphine | Pain |
| capsule | longum + | | |
| | Lactobacillus casei | | |
| Tablet | Bifidobacterium | Eucalyptol + | Stress |
| | breve + | Terpineol | |
| | Lactobacillus gasseri | | |
| Tablet | Bifidobacterium | Thabine | Sleep disorders |
| | breve | | |
| Sublingual | Streptococcus | Thabine + | Addiction |
| oil | thermophilus | Geraniol | |
| Sublingual | Streptococcus | Codeine | Pain |
| oil | thermophilus | | |
| Sublingual | Streptococcus | Pinene | Addiction |
| oil | thermophilus | | |
| Sublingual | Lactobacillus | Naloxone | Visceral pain |
| oil | rhamnosus | | |
| Gel | Lactobacillus | Codeine + | Abuse |
| capsule | helveticus | Limonene | |
| Gel | Bifidobacterium | Morphine + | Sleep disorders |
| capsule | breve + | Pinene | |
| | Lactobacillus | | |
| | rhamnosus | | |
| Tablet | Lactobacillus | Morphine + | Gastrointestinal |
| | jensenii | Myrcene | motility |
| Tablet | Bifidobacterium | Myrene + | Abuse |
| | longum + | Pinene | |
| | Lactobacillus casei | | |
| Tablet | Bifidobacterium | Linalool | Gastrointestinal |
| | breve + | | motility |
| | Lactobacillus gasseri | | |
| Tablet | Bifidobacterium | Diprenorphine + | Visceral pain |
| | breve + | Nerolidol | |
| | Lactobacillus | | |
| | rhamnosus | | |
| Gel | Lactobacillus | Morphine+ | Visceral pain |
| capsule | jensenii | Caryophyllene | |
| Sublingual | Bifidobacterium | Linalool+ | Addiction |
| oil | breve | Terpinene | |
| Sublingual | Streptococcus | Eucalyptol | Alcohol |
| oil | thermophilus | | dependency |
| Tablet | Bacteroide fragilis + | Phellandrene | Abuse |
| | Clostridium | | |
| Gel | Lactobacillus | Carvacrol + | Alcohol |
| capsule | helveticus | Linalool | dependency |

What is claimed is:

1. A method for treating a condition and/or a symptom associated with an effect of at least one element of the opioid system in an animal, the method comprising administering to said animal (i) a therapeutically effective amount of at least one microorganism and/or of a prebiotic thereof, wherein said microorganism is capable of modulating an effect of at least one element of the opioid system of said animal; and (ii) a therapeutically effective amount of at least one compound capable of modulating an effect of at least one element of the opioid system of said animal.

2. The method of claim 1, wherein said microorganism and said at least one compound are capable of modulating a same said at least one element of said opioid system of said animal.

3. The method of claim 1, wherein said microorganism is capable of modulating a first said element of said opioid system and said at least one compound is capable of modulating a second element of said opioid system, wherein said first element is different from said second element.

4. The method of claim 1, wherein said condition and/or symptom is associated with the gastrointestinal tract.

5. The method of claim 1, wherein said condition and/or symptom is selected from the group consisting of autism spectrum disorder, central opioid deficiency, endorphin deficiency, pain, depression, seizures, stress, appetite, addiction, abuse, sleep disorders, impulsive behavior, alcohol dependency, visceral sensation, visceral pain, visceral motility, gastrointestinal motility and combinations thereof and combinations thereof.

6. The method of claim 1, wherein said at least one element of the opioid system is selected from the group consisting of opioid receptors, endogenous opioids, opioids synthesizing enzymes, opioids degrading enzymes, and combinations thereof.

7. The method of claim 1, wherein said at least one microorganism modulates the expression of mu opioid receptors.

8. The method of claim 1, wherein said compound is selected from the group consisting of heroin, codeine, thabine, Fentanyl, hydrocodone, hydromorphone, Dihydrocodeine, oxymorphol, oxycodone, oxymorphone, metopon, nicocodeine, nicomorphine, dihydrocodeine, dihydromorphine, naloxone, naltrexone, diprenorphine, Meperidine, Methadone and combinations thereof.

9. The method of claim 1, further comprising administering a component selected from the group consisting of heat-shock protein 90 (Hsp90) modulators, extracellular-signal-regulated kinase (ERK1) modulators, extracellular-signal-regulated kinase 2 (ERK2) modulators, Jun N-terminal kinase mitogen-activated protein kinase (JNK MAPK) modulators, p38 MAPK modulators, toll-like receptor 4 (TLR4) modulators, norepinephrine reuptake modulators, serotonin reuptake modulators, cholecystokinin (CCK) modulators, neurokinin modulators and combinations thereof.

10. The method of claim 1, wherein said treating induces the production of neurotransmitters and/or neuromodulators in the enteric nervous system.

11. The method of claim 1, wherein said at least one microorganism comprises a strain selected from the group consisting of Lactobacillus, Bifidobacterium, Prevotella, Bacteroides and combinations thereof.

12. The method of claim 1, wherein said administering said at least one microorganism comprises feeding said animal a food comprising a therapeutically effective amount of said microorganism and/or said prebiotic.

13. The method of claim 1, wherein said administering said at least one microorganism is conducted prior to or simultaneously with said administering said compound.

14. The method of claim 1, comprising multiple administrations of said at least one microorganism and/or said prebiotic and multiple administrations of said compound.

15. The method of claim 1, wherein said at least one microorganism and said at least one compound are provided as a kit.

16. A composition comprising
(i) a therapeutically effective amount of at least one microorganism and/or of a prebiotic thereof, wherein said at least one microorganism is capable of modulating an effect of at least one element of the opioid system, and
(ii) a therapeutically effective amount of at least one compound capable of modulating an effect of at least one element of the opioid system.

17. The composition of claim 40, wherein said compound is an allocentric modulator of a GI tract opioid receptor.

18. The composition of claim 40, wherein said compound is an opioid, and/or an opiate.

19. The composition of claim 40, wherein said compound is selected from the group consisting of heroin, codeine, thabine, Fentanyl, hydrocodone, hydromorphone, Dihydrocodeine, oxymorphol, oxycodone, oxymorphone, metopon, nicocodeine, nicomorphine, dihydrocodeine, dihydromorphine, naloxone, naltrexone, diprenorphine, Meperidine, Methadone and combinations thereof.

20. An animal food comprising the composition of claim 40 and at least one food ingredient.
